(19) United States
(12) Patent Application Publication
(10) Pub. No.: US 2022/0409586 A1
(43) Pub. Date: Dec. 29, 2022

(54) TREATMENT AND PREVENTION OF NEUROINFLAMMATION OR AN INFLAMMATORY BRAIN DISORDER

(71) Applicant: Inflazome Limited, Dublin 2 (IE)

(72) Inventors: Matthew COOPER, Cambridge (GB); Luke Anthony John O'Neill, Dublin 2 (GB)

(21) Appl. No.: 17/775,260

(22) PCT Filed: Nov. 6, 2020
(86) PCT No.: PCT/EP2020/081268

§ 371 Date: May 6, 2022

(30) Foreign Application Priority Data
Nov. 7, 2019 (GB) 1916234.6
Jan. 20, 2020 (GB) 2000806.6
Mar. 13, 2020 (GB) 2003643.0
Mar. 25, 2020 (GB) 2004315.4

(51) Int. Cl. A61K 31/415; A61P 25/06

(52) U.S. Cl. CPC A61K 31/415; A61P 25/06

(57) ABSTRACT

The present invention relates to a compound of formula (I): for use in the treatment or prevention of neuroinflammation or an inflammatory brain disorder.

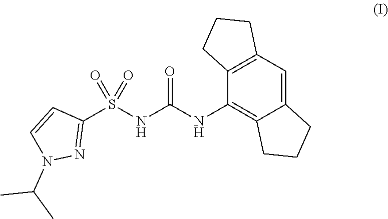 [SMILES: CC(C)n1ccc(S(=O)(=O)NC(=O)Nc2c3c(cc4c2CCC4)CCC3)n1]

The present invention relates to a compound of formula (I):

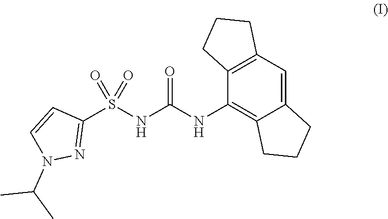 [SMILES: CC(C)n1ccc(S(=O)(=O)NC(=O)Nc2c3c(cc4c2CCC4)CCC3)n1]

for use in the treatment or prevention of neuroinflammation or an inflammatory brain disorder.

Inflammatory brain disorders include Multiple Sclerosis, aseptic meningoencephalitis of autoimmune origin, and migraine.

This invention is based in part on the discovery that the compound of formula (I) is particularly effective in crossing the blood-brain barrier and in inhibiting the NLRP3 inflammatory response in microglia, thus providing effective treatment of neuroinflammation and inflammatory brain disorders. Most especially, neuroinflammation may be effectively inhibited by the oral administration of the compound of formula (I).

In a first aspect of the present invention, there is provided a compound of formula (I):

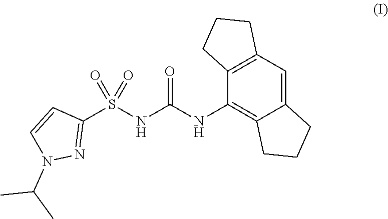 [SMILES: CC(C)n1ccc(S(=O)(=O)NC(=O)Nc2c3c(cc4c2CCC4)CCC3)n1]

or a pharmaceutically acceptable salt thereof, for use in the treatment or prevention of neuroinflammation or an inflammatory brain disorder.

In one embodiment, the compound or salt is for use in the treatment or prevention of an inflammatory brain disorder. In one embodiment the inflammatory brain disorder is Multiple Sclerosis. In another embodiment, the inflammatory brain disorder is aseptic meningoencephalitis of autoimmune origin. In another embodiment, the inflammatory brain disorder is migraine, such as chronic migraine.

In one embodiment, where the compound or salt is for use in the treatment or prevention of an inflammatory brain disorder, the treatment or prevention comprises the treatment or prevention of neuroinflammation. Typically, the treatment or prevention of neuroinflammation is achieved via NLRP3 inhibition. As used herein, the term “NLRP3 inhibition” refers to the complete or partial reduction in the level of activity of NLRP3 and includes, for example, the inhibition of active NLRP3 and/or the inhibition of activation of NLRP3.

In one embodiment, the compound or salt is for use in the treatment or prevention of neuroinflammation. Typically, the treatment or prevention of neuroinflammation is achieved via NLRP3 inhibition.

In one embodiment, the treatment or prevention comprises the oral administration of the compound or the salt thereof. In a further embodiment, the treatment or prevention comprises the once daily oral administration of the compound or the salt thereof.

In one embodiment, the compound or salt is a sodium salt, such as a monosodium salt.

In one embodiment, the compound or salt is a monohydrate. In one embodiment, the compound or salt is crystalline. In one embodiment, the compound or salt is a crystalline monosodium monohydrate salt. In one embodiment, the crystalline monosodium monohydrate salt has an XRPD spectrum comprising peaks at: 4.3° 2θ, 8.7° 2θ, and 20.6° 2θ, all ±0.2° 2θ. In one embodiment, the crystalline monosodium monohydrate salt has an XRPD spectrum in which the 10 most intense peaks include 5 or more peaks which have a 2θ value selected from: 4.3° 2θ, 6.2° 2θ, 6.7° 2θ, 7.3° 2θ, 8.7° 2θ, 9.0° 2θ, 12.1° 2θ, 15.8° 2θ, 16.5° 2θ, 18.0° 2θ, 18.1° 2θ, 20.6° 2θ, 21.6° 2θ, and 24.5° 2θ, all ±0.2° 2θ. The XRPD spectrum may be obtained as described in WO 2019/206871, which is incorporated in its entirety herein by reference.

In one embodiment, the crystalline monosodium monohydrate salt is as described in WO 2019/206871, which is incorporated in its entirety herein by reference. In one embodiment, the crystalline monosodium monohydrate salt has the polymorphic form described in WO 2019/206871, which is incorporated in its entirety herein by reference. In one embodiment, the crystalline monosodium monohydrate salt is prepared according to the method described in WO 2019/206871, which is incorporated in its entirety herein by reference.

Typically, in accordance with any embodiment of the first aspect of the invention, the treatment or prevention comprises the administration of the compound or the salt thereof to a patient. The patient may be any human or other animal. Typically, the patient is a mammal, more typically a human or a domesticated mammal such as a cow, pig, lamb, sheep, goat, horse, cat, dog, rabbit, mouse etc. Most typically, the patient is a human.

In a second aspect of the present invention, there is provided a pharmaceutical composition comprising a pharmaceutically acceptable excipient and a compound or salt of the first aspect of the present invention. In one embodiment, the pharmaceutical composition is suitable for oral administration.

In a third aspect of the present invention, there is provided a method for the treatment or prevention of neuroinflammation or an inflammatory brain disorder in a patient in need thereof, wherein the method comprises administering to the patient in need thereof a therapeutically or prophylactically effective amount of a compound of formula (I):

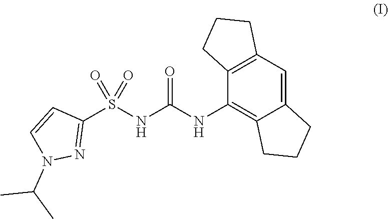 [SMILES: CC(C)n1ccc(S(=O)(=O)NC(=O)Nc2c3c(cc4c2CCC4)CCC3)n1]

or a pharmaceutically acceptable salt thereof.

In one embodiment, the method is a method for the treatment or prevention of an inflammatory brain disorder. In one embodiment the inflammatory brain disorder is Multiple Sclerosis. In another embodiment, the inflammatory brain disorder is aseptic meningoencephalitis of autoimmune origin. In another embodiment, the inflammatory brain disorder is migraine, such as chronic migraine.

In one embodiment, where the method is a method for the treatment or prevention of an inflammatory brain disorder, the treatment or prevention comprises the treatment or prevention of neuroinflammation. Typically, the treatment or prevention of neuroinflammation is achieved via NLRP3 inhibition.

In one embodiment, the method is a method for the treatment or prevention of neuroinflammation. Typically, the treatment or prevention of neuroinflammation is achieved via NLRP3 inhibition.

In one embodiment, the treatment or prevention comprises the oral administration of the compound or the salt thereof. In a further embodiment, the treatment or prevention comprises the once daily oral administration of the compound or the salt thereof.

In one embodiment, the compound or salt is a sodium salt, such as a monosodium salt. In one embodiment, the compound or salt is a monohydrate. In one embodiment, the compound or salt is crystalline. In one embodiment, the compound or salt is a crystalline monosodium monohydrate salt. In one embodiment, the crystalline monosodium monohydrate salt has an XRPD spectrum comprising peaks at: 4.3° 2θ, 8.7° 2θ, and 20.6° 2θ, all ±0.2° 2θ. In one embodiment, the crystalline monosodium monohydrate salt has an XRPD spectrum in which the 10 most intense peaks include 5 or more peaks which have a 2θ value selected from: 4.3° 2θ, 6.2° 2θ, 6.7° 2θ, 7.3° 2θ, 8.7° 2θ, 9.0° 2θ, 12.1° 2θ, 15.8° 2θ, 16.5° 2θ, 18.0° 2θ, 18.1° 2θ, 20.6° 2θ, 21.6° 2θ, and 24.5° 2θ, all ±0.2° 2θ. The XRPD spectrum may be obtained as described in WO 2019/206871, which is incorporated in its entirety herein by reference.

In one embodiment, the crystalline monosodium monohydrate salt is as described in WO 2019/206871, which is incorporated in its entirety herein by reference. In one embodiment, the crystalline monosodium monohydrate salt has the polymorphic form described in WO 2019/206871, which is incorporated in its entirety herein by reference. In one embodiment, the crystalline monosodium monohydrate salt is prepared according to the method described in WO 2019/206871, which is incorporated in its entirety herein by reference.

In accordance with any embodiment of the third aspect of the invention, the patient may be any human or other animal. Typically, the patient is a mammal, more typically a human or a domesticated mammal such as a cow, pig, lamb, sheep, goat, horse, cat, dog, rabbit, mouse etc. Most typically, the patient is a human.

### EXPERIMENTAL

### Figures

FIG. 1: Study A—Levels of the compound of formula (I) in the MetaQuant dialysate from the left striatum of healthy freely-moving adult male mice following oral administration of 1 or 20 mg/kg of the compound (mean+SEM, n=4 per group).

FIG. 2: Study A—Levels of the compound of formula (I) in the MetaQuant dialysate from the right striatum of healthy freely-moving adult male mice following oral administration of 1 or 20 mg/kg of the compound (mean+SEM, n=4 per group).

FIG. 3: Study B—The compound of formula (I) (CPD) displays higher potency than MCC950 in inhibiting NLRP3 inflammasome in primary microglia. A) Dose-dependent inhibition of ATP-induced NLRP3 inflammasome activation in primed microglia by the NLRP3 inhibitor MCC950. The IC₅₀ of MCC950 inhibition with ATP (5 mM) was determined to be 7.5 nM for primary mouse microglia. B) Dose-dependent inhibition of ATP-induced NLRP3 inflammasome activation in primed microglia by the compound of formula (I) (CPD). The IC₅₀ of inhibition with ATP (5 mM) for the compound of formula (I) was determined to be 4.74 nM for primary mouse microglia.

FIG. 4: Study C—Dose-dependent inhibition of ATP-induced NLRP3 inflammasome activation in primed human microglia by the compound of formula (I) (CPD). The IC₅₀ of inhibition with ATP (5 mM) for the compound of formula (I) was determined to be 142 nM for primary human microglia isolated from one healthy donor. Data represent n=1 healthy donor and n=4 technical replicates. Error bars SEM.

### STUDY A—BLOOD-BRAIN BARRIER PENETRATION IN HEALTHY MICE

### Objective

The present study was designed to determine the free concentration of the compound of formula (I) in the left and right striatum of freely-moving adult male mice after oral administration.

### Animals

Adult male C57Bl/6 mice (22-28 g; Envigo, the Netherlands) were used for the experiments. Following arrival, animals were housed in groups of 5 in polypropylene cages (40×50×20 cm) with wire mesh top in a temperature (22±2° C.) and humidity (55±15%) controlled environment on a 12 hour light cycle (07.00-19.00). Following surgery, animals were housed individually (cages 30×30×40 cm). Standard diet (SDS Diets, RM1 PL) and domestic quality mains water were available ad libitum.

### Surgery

Mice were anesthetized using isoflurane (2% and 500 mL/min O₂). Before surgery, Finadyne (1 mg/kg, s.c.) was administered for analgesia during surgery and the post-surgical recovery period. A mixture of bupivacaine and epinephrine was used for local analgesia of the incision site.

### Microdialysis Probe Implantation

The animals were placed in a stereotaxic frame (Kopf instruments, USA). MetaQuant microdialysis probes with a 3 mm exposed polyacrylonitrile membrane (MQ-PAN 3/3) were implanted bilaterally into the left and right striatum (coordinates for the tip of the probe: AP=+0.8 mm (to bregma), ML=+/−1.7 mm (to midline), DV=−4.0 mm (to dura) with an angle of 0° and the incisor bar set at 0.0 mm. All coordinates were based on “The mouse brain in stereotaxic coordinates” by Paxinos and Franklin (2008). The probes were attached to the skull with a stainless-steel screw and dental cement.

### Dose Formulations

The monosodium salt of the compound of formula (I) was formulated in sterilized tap water at concentrations (with respect to the non-salt form) of 0.2 and 4 mg/mL for oral dosing at 5 mL/kg; 1 mg/kg and 20 mg/kg, respectively. The dose formulations are shown in Table 1. The administered volumes for each animal are shown in Table 2.

TABLE 1
Dose formulations

| Formulation | Monosodium salt amount | Solvent |
|---|---|---|
| A | 1.31 mg | 6.19 mL sterilised tap water |
| B | 1.81 mg | 0.428 mL sterilised tap water |
| C | 2.39 mg | 0.565 mL sterilised tap water |

TABLE 2

| Compound administrations | | | |
|---|---|---|---|
| 1 mg/kg | | | |
| | Weight | | Volume |
| Mouse ID | (g) | Formulation | administered (mL) |
| 2015341-364-3530 | 22 | A | 0.11 |
| 2015341-362-3532 | 28 | A | 0.14 |
| 2015341-363-3531 | 22 | A | 0.11 |
| 2015341-366-3528 | 28 | A | 0.14 |

20 mg/kg

| | Weight | | Volume |
|---|---|---|---|
| Mouse ID | (g) | Formulation | administered (mL) |
| 2015341-365-3529 | 25 | B | 0.13 |
| 2015341-367-3495 | 25 | B | 0.13 |
| 2015341-379-3488 | 27 | C | 0.14 |
| 2015341-378-3489 | 25 | C | 0.12 |

### Experimental Design

The MetaQuant microdialysis probes were connected with flexible PEEK tubing (Western Analytical Products Inc. USA; PK005-020) to a microperfusion pump (Harvard) and perfused with a slow flow of artificial CSF (perfusate), containing 147 mM NaCl, 3.0 mM KCl, 1.2 mM CaCl₂, and 1.2 mM MgCl₂, at a flow rate of 0.12 μL/min and a carrier flow of UP+0.02 M FA+0.04% ascorbic acid at 0.8 μL/min. After a minimum of two hours of prestabilisation, microdialysis samples were collected in 60 minute intervals. Following collection of two baseline samples, the compound of formula (I) (1 or 20 mg/kg in sterilised tap water) was administered orally at t=0 minutes. The specific microdialysis sampling schedule is shown in Table 3. Samples were collected into mini-vials (Microbiotech/se AB, Sweden; 4001029) using an automated fraction collector (UV 8301501, TSE, Univentor, Malta). At the end of the experiment, the animals were sacrificed.

TABLE 3
Microdialysis sampling schedule

| Sample | Time | |
|---|---|---|
| number | (min) | Action |
| A1 | | Flow check (30 min sample)-no analysis |
| A2 | | Flow check (30 min sample)-no analysis |
| 101 | −60 | Basal sample |
| 102 | 0 | Basal sample; administer compound after this sample has |
| | | been taken, at t = 0 min |
| 103 | 60 | |
| 104 | 120 | |
| 105 | 180 | |
| 106 | 240 | |
| 107 | 300 | |
| 108 | 360 | |
| 109 | 420 | |
| 110 | 480 | Turn slow flow off after this sample, wait 15 min |
| A3 | 525 | Flow check (30 min sample)-no analysis |
| A4 | 555 | Flow check (30 min sample)-no analysis |

### Bioanalysis

Microdialysate samples from MetaQuant probes contained a nominal volume of 55.2 μL dialysate. Levels of the compound of formula (I) in MetaQuant microdialysate samples were quantified by LC-MS/MS.

The dialysate samples were mixed with acetonitrile and an aliquot of this mixture was injected into the LC system by an automated sample injector (SIL-2MAD, Shimadzu, Japan). Calibrators and in-run QC samples were prepared in analytical dialysate of the same composition as the microdialysate samples.

Chromatographic separation of the compound was performed on a reversed phase column (100×3.0 mm, particle size 2.5 μm, Phenomenex) held at a temperature of 40° C. in a gradient elution run, using eluent B (acetonitrile+0.1% formic acid) in eluent A (ultrapurified water+0.1% formic acid) at a flow rate of 0.3 mL/min.

MS analyses were performed using an API 4000 MS/MS system consisting of an API 4000 MS/MS detector and a Turbo Ion Spray interface (both from Applied Biosystems, USA). The acquisitions were performed in positive ionization mode, with ionization spray voltage set at 5.5 kV. The probe temperature was set at 550° C. The instrument was operated in multiple-reaction-monitoring (MRM) mode.

MRM transitions for the analyte are shown in Table 4. Suitable in-run calibration curves were fitted using weighted (1/x) regression and the sample concentrations were determined using these calibration curves. Accuracy was verified by quality control samples after each sample series. Concentrations were calculated with the Analyst™ data system (Applied Biosystems).

TABLE 4
MRM table

| Analyte | Q1 | Q3 |
|---|---|---|
| Compound of formula (I) | 387 | 190 |

### Data Evaluation

Pharmacokinetic data for the compound of formula (I) is presented as concentrations (mean+SEM) in microdialysate, corrected for dilution during the experiment. Pharmacokinetic data for the compound of formula (I) in microdialysate was not corrected for recovery. Results were plotted in Prism 5 for Windows (GraphPad Software).

### Results

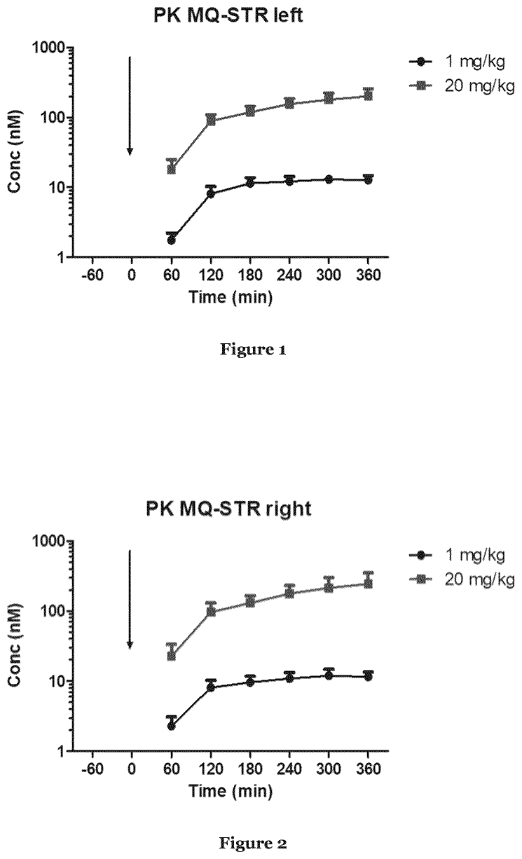

FIG. 1 shows the absolute levels of the compound of formula (I) in the MetaQuant dialysate from the left striatum of freely-moving adult male C57Bl/6 mice following oral administration of 1 or 20 mg/kg of the compound. FIG. 2 shows the absolute levels of the compound of formula (I) in the MetaQuant dialysate from the right striatum of freely-moving adult male C57Bl/6 mice following oral administration of 1 or 20 mg/kg of the compound. 1 mg/kg dosed animals showed average peak levels of 12-13 nM in both the left and right striatal dialysate samples at 5 hours after compound administration. 20 mg/kg dosed animals showed average peak levels of 201-243 nM in both the left and right striatal dialysate samples at 6 hours after compound administration.

As is evident, the results demonstrate the ability of the compound of formula (I) to cross the blood-brain barrier following oral administration. The compound of formula (I) has previously been demonstrated to be a highly effective inhibitor of the activation of the NLRP3 inflammasome (see WO 2016/131098, which is incorporated in its entirety herein by reference). Moreover, inhibition of the NLRP3 inflammasome has been implicated in the treatment of disorders such as Multiple Sclerosis and aseptic meningoencephalitis of autoimmune origin (see for example Masters, Clin Immunol, 2013, 147(3): 223-228; Braddock et al., Nat Rev Drug Disc, 2004, 3: 1-10; Inoue et al., Immunology, 2013, 139: 11-18; and Coll et al., Nat Med, 2015, 21(3): 248-255, all of which are incorporated in their entirety herein by reference). Inhibition of the NLRP3 inflammasome has also been demonstrated to be effective in a mouse model of chronic migraine (see He et al., J Neuroinflammation, 2019, 16: 78, which is incorporated in its entirety herein by reference). As such, it is believed that the compound of formula (I) will be effective in the treatment or prevention of neuroinflammation or inflammatory brain disorders, such as Multiple Sclerosis, aseptic meningoencephalitis of autoimmune origin, and migraine.

### STUDY B—COMPARISON WITH MCC950 IN INHIBITING NLRP3 INFLAMMASOME IN PRIMARY MICROGLIA

### Objective

MCC950 is a previously reported NLRP3 inhibitor (see Coll et al., Nature Medicine, 2015, vol. 21(3), pp. 248-255, which is incorporated in its entirety herein by reference) having the following formula:

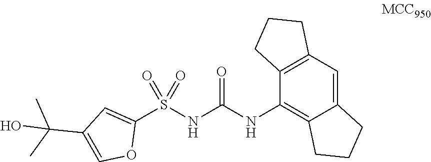 [SMILES: CC(C)(O)c1coc(S(=O)(=O)NC(=O)Nc2c3c(cc4c2CCC4)CCC3)c1]

The aim of study B was to determine the IC₅₀ of the compound of formula (I) and of MCC950 in LPS primed microglia activated with the canonical NLRP3 activator ATP.

### Primary Microglia Cultures

Primary microglial cultures were prepared from C57BL/6 postnatal day 1 (P1) mouse pups and purified by column free magnetic separation system as described previously (see Gordon et al., J. Neurosci. Methods, 2011, vol. 194(2), pp. 287-296, which is incorporated in its entirety herein by reference). Primary microglia were maintained in DMEM/F12 complete medium (DMEM-F12, GIBCO supplemented with 10% heat-inactivated FBS, 50 U/mL penicillin, 50 μg/mL streptomycin, 2 mM L-glutamine, 100 μM nonessential amino acids, and 2 mM sodium pyruvate). Cells were then maintained in a 5% CO₂ incubator at 37° C.

### IL-1β ELISA for IC₅₀ Determination

The mouse IL-1β kit (R&D Systems, Catalog #DY008), was used to measure IL-1β level in the supernatants of LPS primed microglia (3 hours 200 ng/ml) pre-treated with increasing concentrations of MCC950 and the compound of formula (I), and activated with ATP 5 mM for 1 hour.

### Results

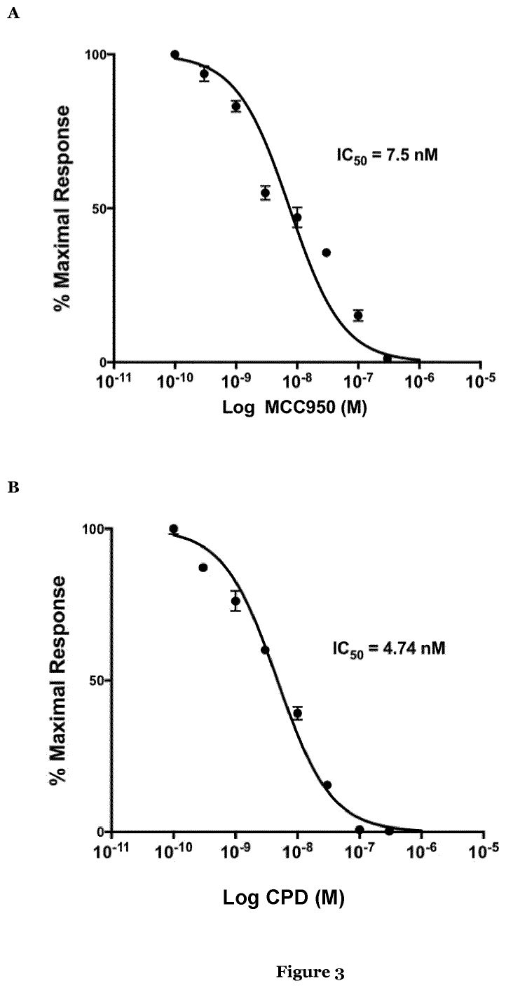

The results are shown in FIG. 3. MCC950 obtained an IC₅₀ of 7.5 nM (FIG. 3A), whereas the compound of formula (I) displayed a potency of 4.7 nM under the same conditions (FIG. 3B). Thus, the compound of formula (I) displays increased potency compared with MCC950 in inhibiting NLRP3 inflammasome in primary microglia.

### STUDY C—INHIBITION OF THE NLRP3 INFLAMMASOME IN PRIMARY HUMAN MICROGLIA

### Objective

To determine the IC₅₀ of the compound of formula (I) in LPS primed human microglia activated with the canonical NLRP3 activator ATP.

### Human Brain Samples

Human brain material was obtained via the rapid autopsy system of the Netherlands Brain Bank (NBB; Amsterdam, the Netherlands), which supplies post mortem material from clinically well-documented and neuropathological confirmed cases and non-neurological controls. Autopsies were performed on donors from whom written informed consent had been obtained by the NBB. One (1) healthy brain tissue sample was used in this experiment.

### Microglia Isolation Method

Human adult microglia cells were isolated and cultured as previously described by Bsibsi et al. (Journal of Neuropathology & Experimental Neurology, 2002, vol. 61(11), pp. 1013-1021). Briefly, at the Netherlands Brain Bank (Amsterdam, The Netherlands), tissue samples were dissected from subcortical white matter and stored in tubes with culture medium at 4° C. The samples were then transported to the laboratory of Charles River Laboratories (Leiden, The Netherlands) in tubes with culture medium. Visible blood vessels were removed and brain tissue was washed with PBS. After a 20-min digestion in 0.25% trypsin the cell suspension was gently triturated and washed with DMEM/HAM-F12 medium containing 10% FCS and antibiotic supplements. After passage through a 100-μm filter, myelin was removed by Percoll gradient centrifugation. Erythrocytes were lysed by 15-min incubation on ice with 155 mM NH₄Cl, 1 mM KHCO₃ and 0.2% BSA in PBS. Next, the cell suspension was seeded into non-coated 96-well plates at a density of 40000-100000 cells/well. To promote proliferation and survival of microglial cells, recombinant human GM-CSF was added to the culture medium at seeding and every 3 days thereafter at a final concentration of 20 ng/ml. After 3-5 days, cultures were washed with medium to remove debris; this was defined as day 0 for the assay. The purity of the cultured microglial cells was verified by immunostaining for microglial identity marker (Iba1) and activation marker (CD45). In addition, cultures were checked for potential contaminating cell populations including astrocytes (GFAP expression) and neurons (NeuN expression). The QC plates were fixed with 4% formaldehyde on the same day of the experiment start.

### IL-1β ELISA for IC₅₀ Determination

At day 0 myelin and cell debris was removed by washing with medium. At day 2 and 3 (T=0 h), culture medium was replaced with 80 μl 100 ng/ml LPS (prepared in serum free medium) to prime microglia. At T=+1.5 h 1000 nM, 200 nM, 40 nM, 8 nM, 1.6 nM, 0.3 nM, 0.064 nM of the compound of formula (I) (in PBS) was added. After 30 min, 5 mM ATP (final concentration, in serum free media) was added to the cultures. At different time points post trigger, supernatants were collected in separate 96-well plates and stored at −20° C. (samples analysed were collected at 2 hour post ATP addition). A Meso Scale Discovery (MSD®) cytokine immunoassay (U-PLEX Human Kit) was used to quantify concentrations of IL-1β in the cell supernatants from each condition, according to manufacturer's instructions provided with the kit (MSD #K151TUK-2). Briefly, MSD plates were coated with capture antibody diluted in Diluent 100 at room temperature for 2 hours on a shaker platform. Plates were washed with 0.05% PBS-Tween, and 25 μL per well of diluent 43 and 25 μL per well of the undiluted samples and standard curve concentrations in technical duplicates were added and incubated overnight at 4° C. while shaking (500 rpm). Plates were washed with 0.05% PBS-Tween, and MSD Sulfo-Tag-conjugated detection antibody diluted in diluent 3 was added to each well and incubated for 1 hour at room temperature while shaking. Plates were then washed with 0.05% PBS-Tween, and 150 μl of MSD Read Buffer-T 4× (with surfactant) diluted 1:2 in water was added to each well. The plates were read using an MSD sector imager model 6000 and the concentration was calculated using MSD discovery Workbench® version 4. Samples were analyzed on an MSD SECTOR S 600 reader and DISCOVERY WORKBENCH analyzed complex set of data generated from MSD plates.

### Results

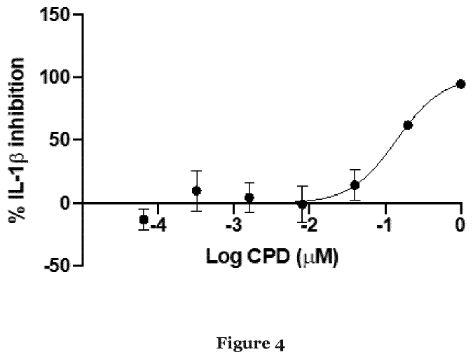

IL-1β concentrations in the supernatants were back-calculated using standard curves of recombinant IL-1β included in the MSD kits. As shown in FIG. 4 the compound of formula (I) obtained an IC₅₀ of 142 nM, thus demonstrating that the compound is effective at inhibiting IL-1β production in human microglia.

Microglia are located in the brain and spinal cord, and act as the main form of active immune defence in the central nervous system. The inflammatory response in microglia is implicated in disorders such as Multiple Sclerosis, aseptic meningoencephalitis of autoimmune origin, and chronic migraine (see for example Luo et al., Neuropsychiatric Disease and Treatment, 2017, vol. 13, pp. 1661-1667; Wang et al., Front. Pharmacol., 2019, vol. 10, article 286; and He et al., J Neuroinflammation, 2019, 16: 78, which are incorporated in their entirety herein by reference). The results presented herein demonstrate both (i) that the compound of formula (I) is a highly potent inhibitor of NLRP3 in microglia, and (ii) that it is able to reach such microglia by crossing the blood-brain barrier following oral administration. As such, it is believed that the compound of formula (I) will be effective in the treatment or prevention of neuroinflammation or inflammatory brain disorders, such as Multiple Sclerosis and aseptic meningoencephalitis of autoimmune origin.

1-17. (canceled)

18. A method for the treatment or prevention of neuroinflammation or an inflammatory brain disorder in a patient in need thereof, wherein the method comprises administering to the patient in need thereof a therapeutically or prophylactically effective amount of a compound of formula (I): 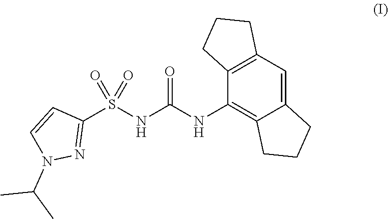 [SMILES: CC(C)n1ccc(S(=O)(=O)NC(=O)Nc2c3c(cc4c2CCC4)CCC3)n1]
or a pharmaceutically acceptable salt thereof.

19. The method as claimed in claim 18, for the treatment or prevention of an inflammatory brain disorder.

20. The method as claimed in claim 19, wherein the inflammatory brain disorder is Multiple Sclerosis.

21. The method as claimed in claim 19, wherein the inflammatory brain disorder is aseptic meningoencephalitis of autoimmune origin.

22. The method as claimed in claim 19, wherein the inflammatory brain disorder is migraine.

23. The method as claimed in claim 19, wherein the treatment or prevention comprises the treatment or prevention of neuroinflammation.

24. The method as claimed in claim 18, for the treatment or prevention of neuroinflammation.

25. The method as claimed in claim 18, wherein the treatment or prevention comprises the oral administration of the compound or the salt thereof.

26. The method as claimed in claim 18, wherein the compound or salt is a sodium salt.

27. The method as claimed in claim 18, wherein the compound or salt is a monosodium salt.

28. The method as claimed in claim 18, wherein the compound or salt is a monohydrate.

29. The method as claimed in claim 18, wherein the compound or salt is crystalline.

30. The method as claimed in claim 18, wherein the compound or salt is a crystalline monosodium monohydrate salt.

31. The method as claimed in claim 30, wherein the crystalline monosodium monohydrate salt has an XRPD spectrum comprising peaks at: 4.3° 2θ, 8.7° 2θ, and 20.6° 2θ, all ±0.2° 2θ.

32. The method as claimed in claim 30, wherein the crystalline monosodium monohydrate salt has an XRPD spectrum in which the 10 most intense peaks include 5 or more peaks which have a 2θ value selected from: 4.3° 2θ, 6.2° 2θ, 6.7° 2θ, 7.3° 2θ, 8.7° 2θ, 9.0° 2θ, 12.1° 2θ, 15.8° 2θ, 16.5° 2θ, 18.0° 2θ, 18.1° 2θ, 20.6° 2θ, 21.6° 2θ, and 24.5° 2θ, all ±0.2° 2θ.

33. The method as claimed in claim 18, wherein the compound or the pharmaceutically acceptable salt thereof is administered as a pharmaceutical composition further comprising a pharmaceutically acceptable excipient.

34. The method as claimed in claim 33, wherein the pharmaceutical composition is suitable for oral administration.
